(19) United States
(12) Patent Application Publication
(10) Pub. No.: US 2024/0123261 A1
(43) Pub. Date: Apr. 18, 2024

(54) RADIATION SHIELDING JIG, METHOD FOR MANUFACTURING THE SAME, AND METHOD FOR USING THE SAME

(71) Applicants: UNIVERSITY OF TSUKUBA, Ibaraki (JP); NATIONAL INSTITUTE OF ADVANCED INDUSTRIAL SCIENCE AND TECHNOLOGY, Tokyo (JP); DAICO MFG CO., LTD., Kyoto (JP)

(72) Inventors: Hiroaki KUMADA, Ibaraki (JP); Naoyuki KITAMURA, Osaka (JP); Tetsuyuki NAKAMURA, Kyoto (JP); Takeshi IKEDA, Kyoto (JP)

(21) Appl. No.: 18/376,923

(22) Filed: Oct. 5, 2023

(30) Foreign Application Priority Data
Oct. 12, 2022 (JP) 2022-164259

(51) Int. Cl. A61N 5/10

(52) U.S. Cl. CPC A61N 5/1077; A61N 5/1031; A61N 2005/1035; A61N 2005/109; A61N 2005/1094

(57) ABSTRACT

The purpose is to prevent the irradiation beam from leaking between the beam irradiation port of the radiation therapy device and the patient affected area that is the target of the emitted irradiation beam, a radiation shielding jig comprising a tare filled with shielding material particles; the tare is made of a resin fabric and has a hollow three-dimensional shape with a radiation pathway portion, the shielding material particles comprising a mixture of sintered particles having a predetermined particle diameter with radiation shielding performance and resin particles having a predetermined particle diameter.

### TECHNICAL FIELD

The present invention relates to a radiation shielding jig, a method for manufacturing the same, and a method for using the same.

In more detail, the present invention relates to the radiation shielding jig, which can improve the positioning accuracy of the patient's affected part in radiation therapy using neutron-containing radiation, reduce the radiation dose leaking from the gap between the radiation emitting port and the patient's affected part, reduce or eliminate the radiation dose irradiated to healthy areas other than the affected part, and reduce the exposure of the human body and peripheral equipment of this treatment device.

### BACKGROUND ART

New applications utilizing the radiation shielding effect of certain elements are being developed in the field of radiation medicine.

Neutrons, one of the most common types of radiation, have no electric charge and are easily absorbed when they collide with atomic nuclei. Absorption of such neutrons is called “neutron capture”, and an example of medical application of this property is “Boron Neutron Capture Therapy: hereinafter referred to as BNCT”. In recent years, the development and practical application of BNCT has been actively pursued in Japan and other major countries, and it is cutting-edge treatment for refractory cancers.

In this BNCT, first, the boron drug containing boron isotope ¹⁰B injected into the body by injection or intravenous infusion is reacted with tumor cells, such as those of malignant cancers, to form the reaction product of the boron compound in this tumor area.

The reaction product is then irradiated with neutrons (should consist primarily of neutrons with moderate energy levels, such as epithermal neutron) in a plane shape at an energy level that has little effect on the healthy parts of the human body, causing a nuclear reaction between the boron compound that has previously been segregated in high concentration in the tumor area and only within a very small area equivalent to one cell, killing only the tumor cells.

Originally, cancer cells are prone to incorporate boron into their tumor cells in the process of proliferating actively.

The BNCT uses this property to effectively destroy only the tumor portion in the treatment.

An irradiation beam consisting mainly of neutrons at an energy level that has little effect on the healthy area is irradiated in a plane shape and sized to encompass the tumor area.

This dramatically reduces irradiation time compared to pinpoint irradiation in conventional radiotherapy. Furthermore, unirradiated areas (irradiation leakage) can be eliminated.

Neutrons have a wide range of energy levels, from high energies above 100 MeV to low energies below 0.002 eV.

In order of energy, they are called “fast neutrons,” “epithermal neutron,” “thermal neutrons,” and so on.

The most desirable of these for the BNCT are “epithermal neutron (energy levels of 0.025 eV to 10 keV).

However, it is difficult to control all of them to epithermal neutron, and the irradiated beam is mixed with fast neutrons with an energy level of 10 keV or higher and thermal neutrons with an energy level of less than 0.025 eV.

Due to the high energy possessed by fast neutrons, fast neutrons mixed in the irradiation beam damage DNA in the cell.

Fast neutrons are absorbed and slowed down by body fluids (the main components are water (H₂O) and nitrogen (N)), which are the main components of the body, and gradually transformed into epithermal neutron.

Epithermal neutron are further transformed into low-energy neutrons below thermal neutrons, but in the process of absorbing the energy, they produce high-energy secondary radiation such as γ-rays.

This secondary radiation, along with fast neutrons, can damage healthy cells, and it will primarily produce delayed side effects, i.e., late adverse events.

On the other hand, the thermal neutrons generated in the absorption and moderation process react with ¹⁰B in the boron compound administered to the affected area to destroy the cancer cells, the so-called neutron capture reaction.

In that case, the active generation of the high-energy secondary radiation will cause side effects on the internal side, i.e., the healthy tissue downstream of the beam flow.

On the other hand, the irradiated thermal neutrons react immediately with the integumentary tissue in the integumentary part, resulting in early adverse events such as inflammation of the skin and loss of hair.

In addition, because neutrons and other types of radiation tend to reflect diffusely on material surfaces, it is not easy to focus the irradiation beam and irradiate the area as set, and areas other than the set area will also be irradiated.

Furthermore, the BNCT method treats a wide variety of diseases, and the body parts to be irradiated are diverse, and the addition of individual differences in body shape makes it difficult to eliminate the gap between the patient's diseased part and the irradiation port.

As a result, part of the irradiation beam leaks out of the system from the gap between the patient's affected area and the irradiation port.

Because of the large dose of the irradiated beam, the amount of radiation leaked, even if only a portion of it, can be significant and cause various problems.

As mentioned above, irradiation beams consist of neutrons and secondary radiation of a wide range of energy levels mixed with high-energy fast neutrons and low-energy thermal neutrons, in addition to highly effective epithermal neutron, and furthermore, it is necessary to take measures that take the above situation into account.

First, as a countermeasure against mixed fast neutrons, it is desirable to absorb their energy with a material containing a high concentration of hydrogen (H), nitrogen (N), or carbon (C) groups, which have large reaction cross-sections for fast neutrons and other high energy level neutrons.

Candidate substances are, for example, water, hydroxyl groups, hydrocarbon (C—H) groups, etc. However, when using the BNCT method, liquids and gases such as water are not easy to handle, making it difficult to apply.

Therefore, it should be a fast neutron countermeasure using a solid hydroxyl group or hydrocarbon (C—H) group at room temperature.

On the other hand, as countermeasures against thermal neutrons, substances such as the radioactive isotope ⁶Li of lithium (Li), ¹⁰B of boron (B), ¹¹³Cd of cadmium (Cd), and ¹⁵⁷Gd of gadolinium (Gd), which have large reaction cross-sections to thermal neutrons, should be considered.

Here, it is necessary to take into account the presence of γ-ray generation as secondary radiation in the absorption process of thermal neutrons.

In the case of ⁶Li, no γ-rays are produced, but in the case of ¹⁰B, γ-rays are produced, albeit slightly, and in the case of ¹¹³Cd and ¹⁵⁷Gd, substantial amounts of γ-rays are produced.

Therefore, ⁶Li can be used without any problem, but γ-ray generation countermeasures are required for materials after ¹⁰B, ¹¹³Cd and ¹⁵⁷Gd.

In addition to those originally mixed in the irradiated beam, there coexist neutrons that are moderated in the process of entering the body as a beam, e.g., irradiated, and become thermal neutrons from high energy and medium energy neutrons such as epithermal neutron.

If the amount originally contaminated is high, the amount of contamination should be kept low, because, as noted above, it causes early adverse events in the patient's outer skin.

On the other hand, the thermal neutrons produced by sequential moderation in the body of the latter high- and medium-energy neutrons are involved in the therapeutic principle of the BNCT method, in which nuclear reactions are induced in boron compounds accumulated in cancer cells to selectively kill those cancer cells.

Therefore, it cannot be said that it should be restricted in general, since it is both beneficial and detrimental depending on its absolute amount and irradiation position.

In considering the various countermeasures required for irradiated beams after the above-mentioned ejection, the factor to be considered is to control beams leaking outside the irradiation target area by using materials that have effective moderation and absorption capabilities for both high-energy fast neutrons and thermal neutrons mixed into the beam in the wide neutron energy range of the irradiated beam.

A typical shielding material for the current BNCT device is “LiF-containing polyethylene resin,” which is used, for example, in the part constituting the irradiation port called the “collimator” of the device, as shown in Patent Document 5. This “LiF-containing poly-ethylene resin” is made by mixing and suspending LiF powder in polyethylene, and the general mixing ratio is 50 wt. % LiF and 50 wt. % polyethylene.

Looking at the components of LiF-containing polyethylene resin, the chemical formula of polyethylene is represented by (C₂H₄)n, which is composed of carbon (C) and hydrogen (H). Polyethylene has a shielding performance against high-energy fast neutrons and epithermal neutron, absorbing some or all of the neutron energy, and has a particularly remarkable shielding performance against fast neutrons.

On the other hand, LiF, as mentioned above, has shielding performance against thermal neutrons and is an excellent shielding material that produces almost harmless alpha rays as secondary radiation to the human body but not harmful gamma rays. Thus, the “LiF-containing polyethylene resin” attempts to slow down and shield neutrons in the entire energy range by sharing the effect of two different components against neutron beams with a wide energy distribution.

However, the actual shielding performance of LiF-containing polyethylene resin has the following problems, resulting in inadequate shielding.

First, the first problem is that LiF-containing polyethylene resin is in a solid state at room temperature and lacks fluidity and flexibility.

As shown in the Patent Documents 1-4 below, this “solid state at room temperature and lack of fluidity and flexibility” is the biggest drawback in terms of functionality when implementing the main application of the present invention, “for preventing gaps between the patient's affected part and the beam irradiation port”.

The second problem is the lack of shielding performance due to the fact that the mixing ratio of LiF and polyethylene is 50 wt. % and 50 wt. %, and the same mixing ratio is used for all parts, especially in the latter part inside the shielding material, where the shielding performance against thermal neutrons and other weak energy neutrons is insufficient.

The mixing ratio of LiF and polyethylene is 50 wt. % and 50 wt. %, and all parts have the same mixing ratio.

The energy distribution of neutrons in the irradiation beam gradually becomes more low-energy neutrons as the flow progresses, with “fast neutrons” successively becoming “epithermal neutron,” “thermal neutrons,” etc., and “epithermal neutron” becoming “thermal neutrons,” etc., and “thermal neutrons” becoming “cold neutrons,” etc.

However, the shielding material, polyethylene resin containing LiF, has the same mixing ratio in all parts, so its shielding performance against low-energy neutrons is insufficient, especially in the latter half of the beam flow, causing low-energy neutrons to leak out.

The third problem is that the existing LiF powder in LiF-containing polyethylene resin has particle sizes of various sizes and lacks shielding performance due to its non-uniform distribution state, especially for low-energy neutrons.

As mentioned above, “LiF-containing polyethylene resin” is made by mixing and suspending powdered LiF in polyethylene, and it is also strongly presumed that the LiF powder has a variety of particle sizes, large and small, and is unevenly distributed.

In the technical field of powder engineering, it is said that when a polymeric material such as resin is mixed with a powder such as LiF powder, the LiF fines on the powder side first aggregate with each other and tend to produce secondary particles, which inevitably results in a non-uniform mixture.

Furthermore, the inventors heated the “LiF-containing polyethylene resin” material on the market at a low temperature, selectively fumigated the resin within it, and investigated the particle size distribution of the LiF powder in the residue. It was inferred that the original LiF raw material with a wide particle size distribution, as well as this one, was used as is.

From the results of this investigation, it is strongly analogous that the distribution of LiF powder in “LiF-containing polyethylene resin” is non-uniform, which is a major cause of the “insufficient shielding performance against low-energy neutrons” mentioned above.

Furthermore, we found that if the mixing ratio of “LiF powder” is increased to solve the second problem above, the inhomogeneity of the distribution of this “LiF powder” becomes stronger and the shielding performance cannot be improved, which is not a solution.

In addition, devices shown in the following Patent Documents 1-4, for example, have been devised as countermeasures to the problems required between the irradiated beam after emission and the patient's affected area, which is the target of the beam, in general radiation therapy devices.

The device described in Patent Document 1 attempts to prevent radiation leakage by arranging plate-like shielding materials called “electron beam guides” with shielding performance in the form of shielding walls and controlling the length of the plate-like materials.

The device described in Patent Document 2 attempts to prevent radiation leakage by providing a skirted cylinder called a “tubes” with shielding performance.

The device described in Patent Document 3 attempts to close the gap by attaching and detaching a cylindrical member called an “adapter” that has shielding performance.

In all of the devices described in the above Patent Document 1-3, various measures are taken to close the gap between the radiation beam irradiation port and the patient's affected area. However, in common, the devices have a rigid structure that gives a feeling of oppression, lacks controllability, and cannot completely close the gap, and none of them use materials with excellent shielding performance, resulting in extremely poor shielding performance.

On the other hand, in the device described in Patent Document 4, a shielding jig made of an inorganic material having shielding performance and a thermoplastic resin is placed between the beam irradiation port and the patient's affected area.

In manufacturing this shielding jig, based on the three-dimensional shape data between the beam irradiation port and the patient's affected part collected in the preliminary examination stage, the thermoplastic resin is heated, molded, cooled, and solidified in an off-line process to make a shielding jig that follows the external shape of the patient's affected part.

Polyolefin is employed as the thermoplastic resin, more specifically polyethylene, and lithium fluoride (LiF) is mixed with this thermoplastic resin as the material used.

It has the same composition as the “LiF-containing polyethylene resin” used in the “collimator” in the device described in the aforementioned Patent Document 5.

Although not specified in the literature, since the melting points of the above resin and LiF are very different, and moreover, it is difficult to melt and uniformly mix the resin and inorganic material LiF together, it is assumed that the LiF powder is simply mixed into the resin, similar to the “LiF-containing polyethylene resin” mentioned above, in other words, it is strongly presumed that the LiF powder is aggregated in the resin.

Therefore, it can be said that there are no remarkable features in terms of material composition, and like the “LiF-containing polyethylene resin” described above, it has various shortcomings.

The shielding jig is set between the beam irradiation port and the patient's affected area (this state is called the “setting state”) as a “solid, or inflexible object” after being molded and solidified, and cannot be used to solve the following problem (5). In other words, it is not “inch-moveable,” and as a result, a gap is created, resulting in beam leakage.

Furthermore, it does not take advantage of the characteristic of thermoplastic resin, which is that it becomes flexible when heated. In other words, it is no different from the shielding structure using solid materials in the above-mentioned Patent Documents 1-3, which have various drawbacks.

The device described in the Patent Document 5 is a typical device configuration for the BNCT method. Polyethylene resin containing LiF, which is said to be the best shielding material among existing materials, is used as the material for the beam narrowing mechanism called a “collimator” that constitutes the beam irradiation port.

However, there is no description of any measures to prevent beam leakage between the beam irradiation port and the patient's affected area, which is the most important problem in the present invention.

### Problem to be Solved by the Invention

In order to enhance the therapeutic effect of BNCT as described above, the main problem is that exist between the irradiated beam after emission and the patient affected area, which is the target of the beam, are as follows.
(1) It is not easy to narrow down the irradiation beam to a set range, and the irradiation beam tends to leak out of the set range, and this leakage of the irradiation beam must be prevented.
(2) No gap shall be created between the patient's affected area and the irradiation port.
(3) The beam irradiation time in the BNCT method is approximately 30 to 60 minutes, but the patient is forced to stay in the same position before and after the irradiation time. The patient's posture should be comfortable with as little pressure as possible, and the method of fixing the patient's affected part should be such that the patient can easily maintain the posture.
(4) In the BNCT method of treatment, the patient's affected area and the beam irradiation port are positioned in advance before the treatment (hereinafter referred to as “setting”), the treatment plan is created using the setting data, and the treatment is started by returning the affected area to the setting state again. The aim is to improve the accuracy of the reproducibility of this setting state.

Regarding the above problem (2), i.e., “to prevent gaps between the patient's affected area and the irradiation port,” a close observation of the actual conditions during treatment reveals that, as described in the problem (3), the irradiation time is approximately 30 to 60 minutes, but the patient is required to maintain the posture in the setting state before and after the irradiation time, for example, during the setup process, which places a long-time burden on the patient.

Thus, including during the irradiation time,
(5) The patient's affected part must be able to be moved slightly (i.e., “inch-able”), and even in the inch-able state, there must be no gap between the affected part and the patient's body.

Furthermore, the first three problems listed above can be identified as specific problems related to shielding performance when LiF-containing polyethylene resin is used as a shielding material.

### PRIOR ART DOCUMENT

### Patent Document

Patent Document 1: Japanese Utility Model Application Laid-Open Publication No. 61-191056
Patent Document 2: Japanese Utility Model Application Laid-Open Publication No. 51-162585
Patent Document 3: Japanese Utility Model Application Laid-Open Publication No. 5-65353
Patent Document 4: Japanese Patent Application Laid-Open Publication No. 2018-15148
Patent Document 5: Japanese Patent Application Laid-Open Publication No. 2009-189643

### SUMMARY OF THE INVENTION

Solution to Problem and Advantageous Effect of Invention The present invention was made in view of the above problems, the purpose of this invention is to provide a radiation shielding jig that can improve the accuracy of patient positioning in radiation therapy using neutron-containing radiation, for example, Boron Neutron Capture Therapy (BNCT), which is attracting attention as a next-generation treatment method for refractory cancer.

Furthermore, it is to provide a radiation shielding jig that can reduce the radiation dose leaking from the gap between the radiation port and the affected area, reduce or eliminate the radiation dose irradiated to healthy areas other than the affected area, and reduce the exposure to the human body including patients and medical personnel, and to peripheral equipment of this treatment device.

Furthermore, it is to provide a method for its production and a method for its use in radiotherapy.

In order to solve various problems related to conventional radiation shielding, the inventors have already invented a shielding material (Japanese Patent Application No. 2021-115328) that far exceeds the shielding performance of conventional shielding materials.

Using the shielding materials of this earlier application, or other shielding materials, and furthermore, based on a new idea that can effectively demonstrate the characteristics of these materials, the present invention has been completed.

In order to solve the above problems (1) to (5), which are required between the irradiated beam and the patient's affected area, we propose, for example, to place a ring-shaped radiation shielding jig at the outer edge of the therapeutic beam entrance of the BNCT system.

The radiation shielding jig is composed of two types of shielding material particles with excellent radiation shielding performance and excellent flowability mixed and filled into a tare with a hollow three-dimensional shape, with an internal atmosphere gas volume adjustment function and a flexible structure that allows the external shape of the jig to be fixed.

The first problem mentioned above in “LiF-containing polyethylene resin,” which is an existing typical shielding material, namely, “it is in a solid state at room temperature and lacks fluidity and flexibility,” is solved by, for example, making a radiation shielding jig filled with shielding material particles with excellent fluidity, which are a mixture of sintered particles and resin particles in a flexible structure tare.

The second problem mentioned above, i.e., “insufficient shielding performance against low-energy neutrons,” is solved, for example, by providing a tare structure that, on top of the bulkheads that ring out the tare above, further provides bulkheads in layers and allows the mixing ratio of both shielding material particles filled in each divided space portion to be adjusted as desired.

The third problem mentioned above, i.e., “insufficient shielding performance against low-energy neutrons due to non-uniform distribution of LiF powder,” is solved by, for example, once the raw material rich in shielding performance against low-energy neutrons is made into a highly homogeneous sintered body, then coarsely fractured, abrasion fractured and sieved to make sintered particles, which are mixed with resin particles to make shielding material particles.

In order to achieve the above object, a radiation shielding jig according to a first aspect of the present invention is characterized by comprising a tare filled with shielding material particles;
the tare is made of a resin fabric and has a hollow three-dimensional shape with a radiation pathway portion; and
the shielding material particles comprising a mixture of sintered particles having a predetermined particle diameter with radiation shielding performance and resin particles having a predetermined particle diameter.

The term “shielding material particles” refers to particles that include “sintered particles” having excellent shielding performance against thermal neutrons and “resin particles” having excellent shielding performance against high-energy neutrons.

“Sintered particles” here refers to particles obtained by fracturing, abrasion fracturing, and sieving sintered body, and “sintered particles” become rounded-shaped, highly fluid particles by fracturing, abrasion fracturing, and sieving.

“Resin particles” are also formed into particles with a predetermined diameter and are highly flowable.

Using the radiation shielding jig according to the first aspect of the invention, even if an irradiation beam leaks out, the energy of the leaked beam can be reduced and absorbed by the shielding material particles to prevent the occurrence of the problems, and the above problem (1) can be solved.

In addition, the radiation shielding jig can have an excellent shape fitting function that allows it to follow the shape of the patient's affected area, the above problem (2) can also be solved by preventing the occurrence of gaps.

The above problem (3) can also be solved by having shielding material particles of a shape with excellent flowability, and a flexible structure tare with high deformation performance, to facilitate the fixation of the shape along the patient's affected area.

In addition, by having shielding material particles of a shape with excellent flowability, and a flexible structure tare with high deformation performance, to facilitate fixation of the shape along the patient's affected area, the above problem (4) “to improve the accuracy of reproducibility of the setting state” can also be solved.

In addition, by having shielding material particles with excellent flowability, and a flexible tare with high deformability, even if the patient moves slightly, the tare can be easily deformed according to the patient's slight movement.

As a result, the occurrence of gaps can be prevented, and the above problem (5) “The patient's affected part must be able to be moved slightly (i.e., “inch-able”), and even in the inch-able state, there must be no gap between the affected part of the patient's body and the radiation shielding jig.” can also be solved.

Also, with regard to the above problem (1), which was assumed when the existing typical shielding material “LiF-containing polyethylene resin” is used between this beam irradiation port and the patient affected area, can also be solved, by making LiF into a homogeneous sintered body once, and then fracturing, abrasion fracturing, and sieving it into sintered particles of a certain particle size, and using spherical particles of a certain particle size for the resin such as polyethylene.

These shielding material particles are solid at room temperature, but have excellent fluidity, and the radiation shielding jig becomes flexible when in contact with the affected patient area, and follows the shape of the patient area, without giving pressure due to immobilization.

The first half of the beam flow is filled with a high concentration of resin particles with excellent shielding performance against high-energy neutrons, and the second half of the beam flow is filled with a high concentration of sintered particles made of LiF or other sintered body with excellent shielding performance against low energy neutrons such as thermal neutrons.

In this way, the lack of shielding performance against thermal neutrons, etc., especially in the shielding material in the latter half can be eliminated, and the above second problem can also be solved.

The above third problem can also be solved, by producing highly homogeneous, high-density sintered LiF in advance, fracturing, abrasion fracturing, and sieving it into sintered particles of a predetermined particle size, and then filling the sintered particles into a tare to eliminate non-uniform distribution state of LiF.

The radiation shielding jig according to a second aspect of the present invention is characterized by at least one ventilation tube with a sealing valve that can be connected to a gas suction pump is connected to the tare according to the first aspects of the present invention.

Using the radiation shielding jig according to a second aspect of the present invention, the amount of atmospheric gas inside the tare can be adjusted via the ventilation tube with a sealing valve, and the external shape of the tare can be fixed more securely.

Therefore, the above problems (3) through (5) can be solved more reliably.

The radiation shielding jig according to a third aspect of the present invention is characterized by having at least one bulkhead with ventilation holes that divides the interior space of the tare into round slices, according to the first or second aspects of the present invention.

The plane that forms the beam irradiation port, i.e., the outer surface of the irradiation port side of the deceleration system of the BNCT device, may be “horizontal” or “vertical”, depending on the constructional style of the BNCT device.

In the case of a “vertical plane”, the hollow three-dimensional tare is used in a vertical position, so the filled shielding material particles tend to move within the tare, and measures are required to prevent this movement.

On the other hand, in the case of a “horizontal plane”, the appearance of the tare is less likely to be deformed by the weight of the tare or shielding material particles.

Therefore, as a measure to prevent the shielding material particles from moving, at least one bulkhead with ventilation holes that divides the interior space of the tare into round slices are provided, and the interior of the tare is divided into multiple compartments to prevent large movement of the shielding material particles.

Using the radiation shielding jig according to a third aspect of the present invention,
the interior space of the tare can be divided into multiple compartments by at least one bulkhead with ventilation holes, which divide the interior space of the tare into round slices pattern, preventing deformation of the tare by its own weight and migration of the shielding material particles. The number of compartments should be at least two or three or more.

The radiation shielding jig according to a fourth aspect of the present invention is characterized by having at least one bulkhead with ventilation holes that divides the interior space of the tare into round slices, according to any one of the first to third aspects of the present invention.

Using the radiation shielding jig according to a fourth aspect of the present invention,
it is possible to fill each layer divided into layers with different mixing ratios of sintered particles and resin particles, so that the layer in the first half corresponding to the upstream side of the beam flow is filled with a high concentration of resin particles with excellent shielding performance against high energy neutrons, while the layer in the second half corresponding to the downstream side is filled with a high concentration of sintered body particles with excellent shielding performance against low energy neutrons such as thermal neutrons, the above second problem can be solved certainly.

The radiation shielding jig according to a fifth aspect of the present invention is characterized by the tare and the resin particles are made of resin selected from polyethylene, polystyrene, and polypropylene, according to any one of the first to fourth aspects of the present invention.

Considering the flexibility of the fabric when formed, its radiation shielding capability, and cost effectiveness, the inventors have examined the use of polyethylene as well as other resins with C—H as a backbone element, such as polystyrene and polypropylene, as resins for the tare and the resin particles, and have confirmed their effectiveness.

Using the radiation shielding jig according to a fifth aspect of the present invention, it is possible to provide a radiation shielding jig with excellent flexibility, radiation shielding capability, and cost performance.

The radiation shielding jig according to a sixth aspect of the present invention is characterized by the bulkhead is made of a resinous fabric selected from polyethylene, polystyrene, and polypropylene, according to any one of the third to fifth aspects of the present invention.

Using the radiation shielding jig according to a sixth aspect of the present invention, similar to a fifth aspect of the present invention, it is possible to provide a radiation shielding jig with excellent flexibility, radiation shielding capability, and cost performance.

The radiation shielding jig according to a seventh aspect of the present invention is characterized by the particle size of the sintered particles and the resin particles are set in the range of 0.5 mm to 7 mm, according to any one of the first to sixth aspects of the present invention.

The tare was filled with shielding material particles of various “particle sizes” and the “flexibility of the jig”, which represents its fluidity, was examined by sensory testing.

It was confirmed that the jig was not flexible when the particle diameter was less than 0.5 mm, and when the particle diameter was coarse, exceeding 7 mm, the patient who came into contact with the jig felt a lumpy and uncomfortable sensation.

Therefore, the appropriate range of “particle size” was set at 0.5 to 7 mm.

Using the radiation shielding jig according to a seventh aspect of the present invention, it is possible to provide a radiation shielding jig that is flexible and does not make the patient feel lumpy or uncomfortable.

The radiation shielding jig according to a eighth aspect of the present invention is characterized by the sintered particles are collected by fracturing and abrasion fracturing and sieving sintered body with a relative density of 70-90%, according to any one of the first to seventh aspects of the present invention.

When the relative density of the original sintered body is higher than 90%, fracturing is not easy. In addition, the shape of most of the fractured particles becomes sharp and blade-like, and the abrasion fracturing time becomes long.

Furthermore, it was confirmed that the production yield of sintered particles begins to decline when the relative density exceeds 82%, and significantly decreases when the relative density exceeds 90%. From these reasons, the upper limit of the relative density was set at 90%.

On the other hand, if the relative density is less than 70%, the mechanical strength of the sintered particles becomes low, and when used as shielding material particles for this application, wear and tear is severe, which may cause problems during repeated use. Therefore, the lower limit of the relative density was set at 70%.

Using the radiation shielding jig according to a eighth aspect of the present invention, it is possible to provide a radiation shielding jig that is easy to manufacture the sintered particles and can withstand repeated use.

The radiation shielding jig according to a ninth aspect of the present invention is characterized by the sintered particles are collected from LiF sintered body, according to the eighth aspects of the present invention.

The LiF sintered body contains a high concentration of Li, which has a large absorption cross-section for neutrons, and have excellent shielding performance against radiation, especially against low-energy neutrons such as thermal neutrons.

Using the radiation shielding jig according to a ninth aspect of the present invention, the sintered particles can be made to have excellent shielding performance against low-energy neutrons such as thermal neutrons.

The radiation shielding jig according to a tenth aspect of the present invention is characterized by the sintered particles are collected from a mixed system sintered body consisting of LiF with a boron compound 0.1-5 wt. % as boron isotope ¹⁰B, wherein a boron compound is selected from B₂O₃, B(OH)₃, BF₃, LiB₃O₅ or Li₂B₄O₇, according to the eighth aspects of the present invention.

Using the radiation shielding jig according to a tenth aspect of the present invention, the sintered particles contain a high concentration of B (isotope ¹⁰B), which has a larger absorption cross-section for neutrons than Li, further making the sintered particles superior in shielding performance against neutrons.

The radiation shielding jig according to a eleventh aspect of the present invention is characterized by the sintered particles are collected from multicomponent system fluoride sintered body with LiF as a main phase, wherein multicomponent system fluoride sintered body containing 99 wt. % to 5 wt. % of LiF and 1 wt. % to 95 wt. % of one or more fluorides selected from MgF₂, CaF₂, AlF₃, KF, NaF and/or YF₃, according to the eighth aspects of the present invention.

Since LiF is a typical hard-to-sinter raw material, it is desirable to select one or more of the other fluorides, such as MgF₂, CaF₂, AlF₃, KF, NaF and/or YF₃, and mix them with LiF, rather than LiF alone, in order to achieve stable and homogeneous sintering.

The reason for selecting other fluorides as substances to be mixed with LiF is that fluorides of the same type as LiF can easily form a solid solution in the sintering process, which generates an eutectic point and lowers the sintering temperature.

As a result, the decomposition and vaporization (i.e., sublimation) phenomenon of fluorides such as LiF is suppressed, which prevents foaming and makes it easier to obtain dense sintered compacts.

Using the radiation shielding jig according to a eleventh aspect of the present invention, it is easy to produce sintered particles that contain a high concentration of Li, which has a large absorption cross-section for neutrons, and that have excellent shielding performance against radiation, especially against low-energy neutrons such as thermal neutrons.

Moreover, high-density, highly homogeneous sintered particles for radiation shielding materials can be provided stably and at low cost.

The radiation shielding jig according to a twelfth aspect of the present invention is characterized by the sintered particles are collected from a mixed system sintered body consisting of multicomponent system fluoride with LiF as a main phase and boron compounds selected from B₂O₃, B(OH)₃, BF₃, LiB₃O₅ or Li₂B₄O₇, with 0.1 to 5 wt. % as boron isotope ¹⁰B added, according to the eighth aspects of the present invention.

Using the radiation shielding jig according to a twelfth aspect of the present invention, the sintered particles contain a high concentration of B (isotope ¹⁰B), which has a larger absorption cross-section for neutrons than Li, further making the sintered particles superior in shielding performance against neutrons.

The radiation shielding jig according to a thirteenth aspect of the present invention is characterized by the sintered particles are collected from a mixed system sintered body consisting of multicomponent system fluoride with LiF as a main phase and a gadolinium compound selected from Gd₂O₃, Gd(OH)₃ or GdF₃, with 0.1 to 2 wt. % as gadolinium isotope ¹⁵⁷Gd added, according to the eighth aspects of the present invention.

In this application, we have also decided to provide excellent sintered particles that contain high concentrations of Li, which has a large absorption cross-section for neutrons, and Gd (isotope ¹⁵⁷Gd), which has a larger absorption cross-section than Li, and that are high-density and highly homogeneous, in a stable and inexpensive manner.

The reason for adding 0.1-2 wt. % of gadolinium compound as ¹⁵⁷Gd to the multicomponent system fluoride raw material was because it was confirmed in tests that the addition of less than 0.1 wt. % of gadolinium compound did not improve the shielding effect, while the addition of more than 2 wt. % reduced the density of the sintered body and made it difficult to maintain the sintered body's shape.

Based on the test results, the appropriate range of gadolinium compound addition was determined to be 0.1 to 2 wt. % as ¹⁵⁷Gd.

Using the radiation shielding jig according to a thirteenth aspect of the present invention, the high concentration of Gd (isotope ¹⁵⁷Gd), which has a larger absorption cross-section for neutrons than Li, can further make the sintered particles superior in neutron shielding performance.

The radiation shielding jig according to a fourteenth aspect of the present invention is characterized by the sintered particles are collected from a mixed system sintered body consisting of multicomponent system fluoride with LiF as a main phase, and boron compounds selected from B₂O₃, B(OH)₃, BF₃, LiB₃O₅ or Li₂B₄O₇, with 0.1 to 5 wt. % as boron isotope ¹⁰B added, and a gadolinium compound selected from Gd₂O₃, Gd(OH)₃ or GdF₃, with 0.1 to 2 wt. % as gadolinium isotope ¹⁵⁷Gd added, according to the eighth aspects of the present invention.

In this application, we have also decided to provide excellent sintered particles that contain high concentrations of Li, which has a large absorption cross-section for neutrons, and B (isotope ¹⁰B) and Gd (isotope ¹⁵⁷Gd), which has a larger absorption cross-section than Li, and that are high-density and highly homogeneous, in a stable and inexpensive manner.

The sintered body should have high concentrations of the isotopes ⁶Li, the isotopes ¹⁰B in addition to isotopes ⁶Li, and the isotopes ¹⁵⁷Gd, to achieve the required shielding performance.

Using the radiation shielding jig according to a fourteenth aspect of the present invention, the high concentration of B (isotope ¹⁰B) and Gd (isotope ¹⁵⁷Gd), which have a larger absorption cross-section for neutrons than Li, further makes the sintered particles excellent in neutron shielding performance.

The radiation shielding jig according to a fifteenth aspect of the present invention is characterized by the sintered particles of which are formed by fracturing, abrasion fracturing, and sieving the sintered body, are not collected particles, using the not collected particles, the sintered particles are formed by re-pulverizing, mixing with the raw powder, and re-sintering, according to any one of the first to fourteenth aspects of the present invention.

Of the above sintered particles, those with particle diameters outside the appropriate range, and of the fractured particles produced in the fracturing process, those with coarse particles or fine powder that cannot be used in the abrasion fracturing process, and those with no impurity contamination or other problems, are pulverized and reused as raw materials for the same composition. From an economic standpoint, this reuse of unused sintered particles becomes an important elemental technology.

Using the radiation shielding jig according to a fifteenth aspect of the present invention, a radiation shielding jig with excellent economic efficiency can be provided.

The radiation shielding jig according to a sixteenth aspect of the present invention is characterized by the mixing ratio of the sintered particles and the resin particles in the shielding material particles is set between 10 wt. % and 90 wt. %, the angle of repose of the shielding material particles is from 8 to 45 degrees of fluidity, according to any one of the first to fifteenth aspects of the present invention.

It is difficult to shield neutron beams with a wide energy distribution, such as the therapeutic beams that are the object of shielding in this application, with only one type of shielding material particles.

Since the mixing ratio of one type of shielding material must be at least 10 wt. %, the shielding material used in the present invention is designed to respond to the energy distribution of the therapeutic beam at the shielding site by setting the mixing ratio of sintered particles and resin particles in the range of 10 wt. % to 90 wt. %.

In order to ensure the fluidity of the shielding material particles, the angle of repose of the shielding material particles was set to from 8 to 45 degrees.

Using the radiation shielding jig according to a fifteenth aspect of the present invention, it can provide sufficient shielding performance even for neutron beams with a wide energy distribution, such as therapeutic beams, and ensure its fluidity and flexibility.

The radiation shielding jig according to a seventeenth aspect of the present invention is characterized by the upstream portion of the beam flow of the tare, which is divided into layers, is filled with the sintered particles in a ratio of not less than 10 wt. % and not more than 50 wt. % and the resin particles in a ratio of not less than 50 wt. % and not more than 90 wt. %, on the other hand, the downstream portion of the beam flow of the tare is filled with the sintered particles in a ratio of not less than 50 wt. % and not more than 90 wt. % and the resin particles in a ratio of not less than 10 wt. % and not more than 50 wt. %, according to any one of the fourth to sixteenth aspects of the present invention.

Using the radiation shielding jig according to a seventeenth aspect of the present invention, the first half of the beam flow is filled with a high concentration of resin particles with excellent shielding performance against high-energy neutrons, while the second half of the beam flow is filled with a high concentration of sintered particles with excellent shielding performance against low-energy neutrons, such as thermal neutrons, in particular, the lack of shielding performance against low-energy neutrons in the latter part of the shielding material is solved, so, the above second problem can be solved.

The first method for manufacturing a radiation shielding jig according to any one of the first to seventeenth aspects of the present invention, is characterized by comprising the steps of:
fracturing the sintered body using a fracturing machine and abrasion fracturing using a abrasion fracturing machine, and sieving, collecting the sintered particles of a predetermined particle size;
mixing the collected sintered particles with resin particles of a predetermined particle size in a predetermined ratio; and
filling the mixed sintered particles and the resin particles into the tare.

According to the first method for manufacturing the radiation shielding jig described above, for example, once raw materials such as LiF are made into a homogeneous sintered body, they are fractured, abrasion fractured, and sieved into sintered particles of a predetermined particle size, while spherical particles of a predetermined particle size are used for resins such as polyethylene, so that these shielding material particles are solid at room temperature, but have excellent flowability.

It is possible to reliably produce a radiation shielding jig that is flexible when in contact with the patient's affected area and follows the shape of the patient's affected area.

It is also easy to fill the first half of the beam flow, corresponding to the upstream side, with a high concentration of resin particles with excellent shielding performance against high-energy neutrons, while the second half, corresponding to the downstream side, is filled with a high concentration of sintered particles made of LiF or other sintered materials with excellent shielding performance against low-energy neutrons such as thermal neutrons.

In particular, it is possible to reliably produce a radiation shielding jig that can eliminate the lack of shielding performance against low-energy neutrons such as thermal neutrons in the latter part of the shielding material.

For example, a highly homogeneous, high-density LiF sintered body or the like can be produced in advance, which is then fractured, abrasion fractured, and sieved into sintered particles of a predetermined particle size, and the sintered particles can then be tare-filled to eliminate the heterogeneous distribution state of LiF.

It is possible to reliably produce a radiation shielding jig that can resolve the state of non-uniform distribution of LiF and solve the lack of shielding performance.

The first method for using a radiation shielding jig according to any one of the first to seventeenth aspects of the present invention, is characterized by comprising the steps of:
placing the patient's affected area to the tare with no gap in the treatment position; and
initiating radiation therapy then.

According to the first method for using the radiation shielding jig described above,
the above problems (1) through (5) can be solved with the use of a radiation shielding jig.

The second method for using a radiation shielding jig according to any one of the second to seventeenth aspects of the present invention, is characterized by comprising the steps of:
opening the sealing valve of the ventilation pipe to which the gas suction pump is connected, and closing the other sealing valves, and placing the patient's affected area to the tare with no gap in the treatment position, while operating the gas suction pump;
in this placing state, closing the sealing valve of the ventilation pipe to which the gas suction pump is connected to fix the external shape of the tare; and
initiating radiation therapy then.

According to the second method for using the radiation shielding jig described above, it is easy to ensure the use of radiation shielding jigs that solve the above problems (1) through (5).

The third method for using a radiation shielding jig according to any one of the second to seventeenth aspects of the present invention, is characterized by comprising the steps of:
opening the sealing valve of the ventilation pipe to which the gas suction pump is connected, and closing the other sealing valves, and placing the patient's affected area to the tare with no gap in the treatment position, while operating the gas suction pump;
in this placing state, closing the sealing valve of the ventilation pipe to which the gas suction pump is connected to fix the external shape of the tare;
measuring the external shape of the fixed tare then;
performing simulation calculations on the behavior of the irradiation beam during treatment based on the measured external shape data of the tare then;
creating a treatment plan based on the results of this simulation calculation; and
performing radiation therapy according to the treatment plan.

According to the third method for using the radiation shielding jig described above, advanced radiotherapy can be performed more reliably and easily according to the treatment plan.

### BRIEF DESCRIPTION OF DRAWINGS

FIG. 1(a) shows a front view of the first Embodiment of radiation shielding jig, and FIG. 1(b) shows a diagonal view.

FIG. 2 shows a side cross-sectional view of the fifth Embodiment of the radiation shielding jig.

FIG. 3 shows a front view of the fifth Embodiment of the radiation shielding jig.

FIG. 4 shows a partial cross-sectional side view of the system including the deceleration system of the BNCT device to illustrate the use of the radiation shielding jig in BNCT.

FIG. 5 shows an illustration of the method of evaluating the shielding performance of radiation shielding jig through simulated testing.

FIG. 6 shows Table 1, which shows the setting of the particle formulation conditions for the shielding layer in the simulation calculations.

FIG. 7 shows Table 2, which shows the results of the simulation analysis.

### DESCRIPTION OF EMBODIMENTS

The preferred Embodiment of the radiation shielding jig, the method for manufacturing the same, and the method for using the same according to the present invention is described below by reference to the Figures.

[Embodiments of Radiation Shielding Jigs]

### The First Embodiment

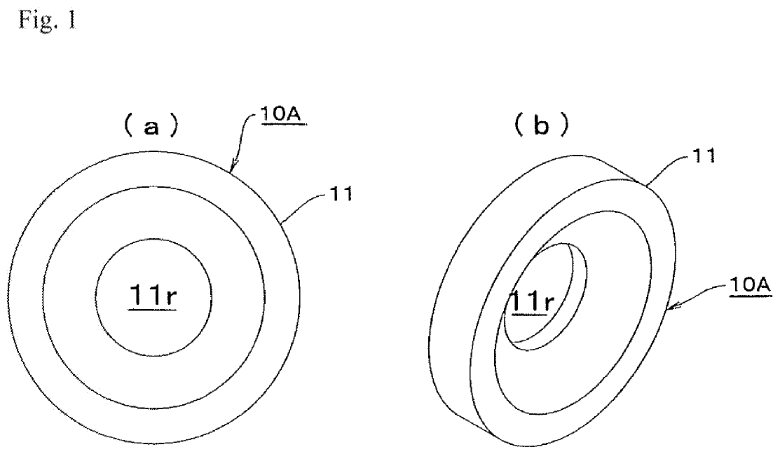

FIG. 1(a) shows a front view of the first Embodiment of radiation shielding jig, and FIG. 1(b) shows a diagonal view.

The radiation shielding jig 10A comprising a tare 11 filled with shielding material particles (not shown), the tare 11 is made of non-woven resin fabric and has a hollow three-dimensional shape with a radiation pathway portion 11r.

The shielding material particles comprising a mixture of sintered particles having a predetermined particle diameter with radiation shielding performance and resin particles having a predetermined particle diameter.

The shielding material particles consist of sintered particles obtained by coarsely fracturing, abrasion fracturing, and sieving the sintered bodies and resin particles, which are filled into the tare 11 so that these particles have equal packing density.

The sintered particles are produced from “sintered bodies with LiF as the main or single phase” that have excellent shielding performance against thermal neutrons.

The resin particles are made of resin selected from polyethylene, polystyrene, and polypropylene, which have excellent shielding performance against fast neutrons and other high-energy neutrons, and have a certain particle size of excellent flowability.

The tare 11, which is filled with shielding material particles, has a hollow three-dimensional shape that surrounds the edge of the beam irradiation opening, which is generally a round opening in the plane (i.e., a circular opening with a diameter of about 100 mm to 250 mm in the plane (most of which are 100 mm to 150 mm in diameter)).

However, the shape of the tare 11 shown in FIG. 1 presents a ring shape, but this shape is not limited to a ring shape in any way, but is determined according to the shape of the area where it will be used.

The tare 11 is also made of a flexible non-woven fabric made of a resin selected from polyethylene, polystyrene, and polypropylene.

The sintered particles are, in more detail, homogeneous, high-density sintered bodies consisting of LiF alone, or a mixture of LiF and a boron compound, or a fluoride system consisting of LiF as the main phase, or a fluoride system consisting of LiF as the main phase with a boron compound, or a fluoride system consisting of LiF as the main phase with a gadolinium compound, or a fluoride system consisting of LiF as the main phase with a boron compound and a gadolinium compound, which have excellent shielding performance against low energy neutrons such as thermal neutrons.

The homogeneous, high-density sintered body is fractured and abrasion fractured and sieved to obtain a particle size and rounded, distinctive particle shape with excellent flow performance.

The following is a description of the “distinctive rounded particle shape” of the sintered particles.

The shape of the fractured particles in the stage of fracturing the sintered body generally has sharp edges. In a simple example, in the Stone Age, when obsidian, a typical stone tool mineral, is coarsely fractured, the configuration is similar to a mixture of coarse particles similar in shape to small pieces with knife-like sharp edges and finely fractured fine powder.

The coarse particles can be collected and abrasion fractured using, for example, a pot mill to selectively abrasion fracture the edges of the coarse particles to produce the sintered particles referred to in the present application, which have a distinctive rounded particle shape.

The term “abrasion fracturing” refers to a fracturing method in which fracturing pressure is applied from multiple directions to exert shear forces mainly on the particle surface layer, for example, to selectively shear the sharp edges of the particles.

Several specific abrasion fracturing methods include stone abrasion fracturing, pot mill abrasion fracturing, and media agitation abrasion fracturing. In this Embodiment, the pot mill abrasion fracturing method is described as an example. The abrasion fracturing method is not limited to this pot mill abrasion fracturing method, but other abrasion fracturing methods with low abrasion fracturing impact as described above can also be applied.

The rounded shape of the sintered particles makes them easier to roll when piled on a flat plate, for example, and the shape of the piled pile (hereinafter referred to as “pile shape”) becomes a gently sloping pile shape, and the angle of repose, which is an indicator of the fluidity of the powder or grain, becomes smaller, thus increasing the fluidity of these particles.

In the present invention, the large flowability of the sintered body particles is one of the key elemental technologies for solving the above problem. In other words, shielding material particles with large flowability consisting of sintered particles and resin particles are filled into a tare 11 having a flexible structure to make a radiation shielding jig 10A with a flexible structure.

This improves the fitting with the patient affected area and prevent irradiation beam leakage without creating gaps between the patient affected area and the radiation shielding jig 10A.

The factors that determine the flowability of the shielding material particles are the size of the particles, or “particle size,” and the shape of the particles, or “particle shape. In the present Embodiment, the latter “particle shape” is a sintered particle with a rounded shape that is consistently fluid, so that its flowability is determined by the remaining “particle size”.

Therefore, we filled the tare 11 with shielding material particles of various “particle sizes” and examined the “flexibility of the jig,” which represents its flowability, by sensory testing.

It was confirmed that the jig was not flexible when the particle diameter was less than 0.5 mm, and when the particle diameter was coarse, exceeding 7 mm, the patient who came into contact with the jig felt a lumpy and uncomfortable sensation.

Therefore, the appropriate range of “particle size” was set at 0.5 to 7 mm.

Next, we will explain how to measure the “average particle diameter” and “angle of repose,” which are the main characteristics of the sintered and resin particles that make up the shielding material particles.

The “average particle size” is measured by sieving using a JIS sieve, and is indicated by its median diameter.

The angle of repose was measured by standing a pair of transparent vinyl chloride plates vertically on a surface plate with a 10 mm gap between them, dropping a predetermined amount of 75 g of each particle into the gap from a single location in the upper center of the gap, and measuring the inclination angle of the mountainous shape of the piled particles.

The tare 11 of the present Embodiment was prepared beforehand, and the tare 11 was filled with shielding material particles of various size distributions and particle shapes, that is, particles with various “angles of repose”, and the “angle of repose” was set within an appropriate range by setting up a simulated patient affected area and sensually inspecting the “flexibility” of the jig, i.e., the “fluidity” of the particles.

As a result, the range from 8 to 45 degrees was determined to be the appropriate range, and the range from 10 to 35 degrees, with a median of 20 degrees, which is an extremely favorable feel, was determined to be the best range.

The larger the particle size and the more spherical the shape, the better the “flowability,” i.e., the smaller the angle of repose. On the other hand, the smaller the particle size and the more angular the shape, the worse the “flowability,” i.e., the larger the angle of repose.

Of the above sintered particles, those with particle diameters outside the appropriate range, and of the fractured particles produced in the fracturing process, those with coarse particles or fine powder that cannot be used in the fracturing process, and those with no impurity contamination or other problems, are pulverized and reused as raw materials for the same composition.

From an economic standpoint, this reuse of unused sintered particles becomes an important elemental technology.

Two types of sintered particles mixed with resin particles in an arbitrary ratio of 10 wt. % to 90 wt. %, i.e., shielding material particles, were filled in the tare 11, with the mixing ratio adjusted.

Specifically, the first half of the beam flow, corresponding to the upstream side, is filled with a high concentration of resin particles with excellent shielding performance against high-energy neutrons, while the second half of the beam flow, corresponding to the downstream side, is filled with a high concentration of sintered particles with excellent shielding performance against low-energy neutrons, such as thermal neutrons.

Thereby, in particular, the lack of shielding performance against low-energy neutrons, such as thermal neutrons, in the latter part of the shielding material is solved.

The mixing ratio of both shielding material particles was set to a ratio suitable for the energy distribution of neutrons in the beam to be shielded.

The reason for setting these upper and lower limits on the mixing ratio is that each of the mixed particles has a distinct role to play with respect to shielding.

In other words, the sintered particles have excellent shielding performance mainly against thermal neutrons, but poor shielding performance against fast and high-energy neutrons such as epithermal neutrons.

On the other hand, resin particles have excellent shielding performance against high-energy neutrons, mainly fast neutrons, but low shielding performance against low-energy neutrons, mainly thermal neutrons.

For this reason, it is difficult to shield neutron beams with a wide energy distribution, such as the therapeutic beams that are the object of shielding in this application, with only one type of shielding material particles.

Since the mixing ratio of one type of shielding material must be at least 10 wt. %, the shielding material used in the present Embodiment is designed to respond to the energy distribution of the therapeutic beam at the shielding site by setting the mixing ratio of sintered particles and resin particles in the range of 10 wt. % to 90 wt. %.

In the present Embodiment, regarding the above problem (1) “It is not easy to narrow down the irradiation beam to a set range, and the irradiation beam tends to leak out of the set range, and to prevent leakage of the irradiation beam,” even if leakage does occur, the shielding material particles in the radiation shielding jig reduce and absorb the energy of the leaked beam to prevent problems.

With regard to the above issue (2) “to prevent gaps from occurring between the patient affected area and the irradiation port,” the radiation shielding jig is designed to have an excellent shape fitting function that allows it to follow the shape of the patient affected area, thereby preventing the occurrence of the above gaps.

With regard to the above problem (3) “to make a method of fixing the patient affected area with less pressure and ease of maintaining the posture,” the above problem was solved by making it easy to fix the shape along the patient affected area by using shielding material particles with excellent flowability and a flexible structure tare with high deformation performance.

With regard to the above problem (4), “Improvement of the accuracy of reproducibility of the setting state,” the above problem was solved by facilitating the fixation of the shape along the patient affected area by using shielding material particles with excellent flowability and a flexible tare structure with high deformation performance, in the same way as the countermeasure for the above problem (3).

With regard to the above problem (5) “The patient's affected part must be able to be moved slightly (i.e., “inch-able”), and even in the inch-able state, there must be no gap between the affected part of the patient's body and the radiation shielding jig.”, in the same way as the countermeasure for the above problem (3), by having shielding material particles with excellent flowability, and a flexible tare with high deformability, even if the patient moves slightly, the tare can be easily deformed according to the patient's slight movement.

As a result, the occurrence of gaps can be prevented, and the above problem (5) can also be solved.

According to the present Embodiment, various problems that are assumed when using an existing typical shielding material, “LiF-containing polyethylene resin,” between this beam irradiation port and the patient's affected area, can also be solved.

Regarding the first problem above, “LiF-containing polyethylene resin is in a solid state at room temperature and lacks fluidity and flexibility”, can be solved, in the present Embodiment, by making LiF into a homogeneous sintered body once, and then fracturing, abrasion fracturing, and sieving it into sintered particles of a certain particle size, and using spherical particles of a certain particle size for the resin such as polyethylene.

Regarding the above second problem, “Insufficient shielding performance due to the mixing ratio of LiF and polyethylene being 50 wt. % and 50 wt. %, and the same mixing ratio in all parts, especially in the latter part inside the shielding material, where the shielding performance against neutrons with weak energy, including thermal neutrons, is insufficient.”, can be solved, in the present Embodiment, by making it possible to adjust the mixing ratio between the upstream and downstream sides of the beam flow by making the sintered LiF particles, which are made by fracturing, abrasion fracturing, and sieving the sintered LiF single phase, and the resin particles made of polyethylene mixed at an arbitrary ratio of 10 wt. % to 90 wt. % as shielding material particles, and thereby making it possible to fill the particles at the upstream and downstream sides of the beam flow.

Specifically, the first half of the beam flow, corresponding to the upstream side, is filled with a high concentration of resin particles with excellent shielding performance against high-energy neutrons, while the second half of the beam flow, corresponding to the downstream side, is filled with a high concentration of sintered particles with excellent shielding performance against low-energy neutrons, such as thermal neutrons.

Thereby, in particular, the lack of shielding performance against low-energy neutrons, such as thermal neutrons, in the latter part of the shielding material is solved.

Regarding the above third problem, “Insufficient shielding performance due to the existing LiF powder in LiF-containing polyethylene resin having various particle sizes and a non-uniform distribution state.”, can be solved, in the present Embodiment, by a highly homogeneous, high-density LiF sintered body was produced in advance, which was fractured, abrasion fractured, and sieved to form sintered particles of a predetermined particle size, and the sintered particles were then filled into the tare to eliminate the uneven distribution state of LiF and solve the lack of shielding performance.

### The Second Embodiment

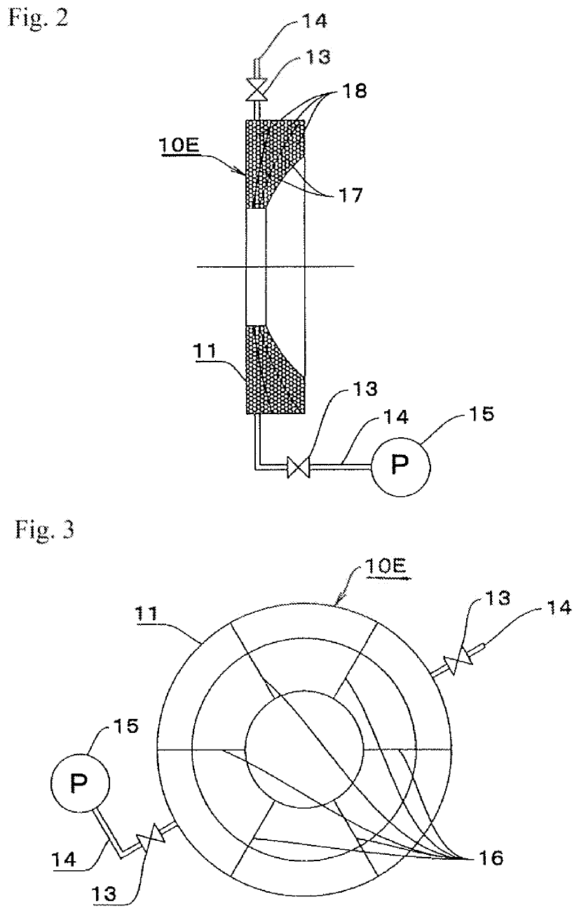

The second Embodiment of radiation shielding jig 10B (not shown) consists of the first Embodiment of radiation shielding jig 10A shown in FIG. 1, with the ventilation pipe 14 with sealing valve 13 shown in FIGS. 2 and 3 connected to opposite sides of the tare 11.

One of these ventilation tube 14 is connected to a gas suction pump 15.

According to the second Embodiment of radiation shielding jig 10B, the adjustment of the amount of atmospheric gas inside the tare 11 can be performed by driving the gas suction pump 15 via the ventilation pipe 14 with sealing valve 13, which ensures that the fixation of the external shape of the tare 11 can be executed reliably.

Therefore, the above problems (3) to (5) can be solved at a higher level, and in particular, it can make a significant contribution to “improving the accuracy of the reproducibility of setting conditions.

### The Third Embodiment

In the third Embodiment of radiation shielding jig 10C (not shown), the radiation shielding jig 10B of the second Embodiment is further provided with a bulkhead 16 with ventilation holes that divide the inside of the tare 11 shown in FIG. 3 into round slices.

By dividing the interior of the tare 11 into multiple compartments into round slices, the shielding material particles 18 can be prevented from moving significantly.

The plane that forms the beam irradiation port, i.e., the outer surface of the irradiation port side of the deceleration system of the BNCT device, may be “horizontal” or “vertical”, depending on the constructional style of the BNCT device.

In the case of a “vertical plane”, the hollow three-dimensional tare 11 is used in a vertical position, so the filled shielding material particles 18 tend to move downward within the tare 11, and measures are required to prevent this movement.

Therefore, as a measure to prevent the shielding material particles 18 from moving, bulkhead 16 with ventilation holes that divides the interior space of the tare 11 into round slices are provided, and the interior of the tare 11 is divided into multiple compartments into round slices to prevent large downward movement of the shielding material particles 18.

In the one shown in FIG. 3, six bulkheads 16 are provided and the interior of tare 11 is divided into six compartments, but the number of compartments should be at least two or three or more.

As described above, when the radiation shielding jig 10C is used in a vertical position, it was feared that the weight of the shielding material particles 18 would deform the tare 11. However, by dividing the inside of the tare 11 into multiple compartments, it is possible to prevent deformation of the radiation shielding jig 10C and shielding material particles 18 from moving due to their own weight.

### The Fourth Embodiment

In the fourth Embodiment of radiation shielding jig 10D (not shown), the radiation shielding jig 10B of the second Embodiment is further provided with a bulkhead 17 with ventilation holes that divide the tare 11 shown in FIG. 2 into layers.

The division of the interior of the tare 11 into multiple layers, prevents large movement of the shielding material particles 18 across the layers.

In the one shown in FIG. 2, two bulkheads 17 are provided and the interior of the tare 11 is divided into three sections in layers, but the number of compartments is at least two, preferably three or more.

According to the radiation shielding jig 10D, it is possible to fill each layer divided into layers with shielding material particles 18 with different mixing ratios of sintered body particles and resin particles.

Filling each layer with a high concentration of resin particles with excellent shielding performance against high-energy neutrons in the layer in the first half of the section corresponding to the upstream side of the beam flow and, on the other hand, with a high concentration of sintered particles with excellent shielding performance against low-energy neutrons such as thermal neutrons in the layer in the second half of the section corresponding to the downstream side is performed.

Therefore, the above second problem, “The mixing ratio of LiF and polyethylene is 50 wt. % and 50 wt. %, and the shielding performance is insufficient due to the fact that the mixing ratio is the same in all parts, especially in the latter part inside the shielding material, where the shielding performance against weak energy neutrons, including thermal neutrons, is insufficient.”, can be solved reliably.

### The Fifth Embodiment

FIG. 2 shows a cross-sectional view of the fifth Embodiment of the radiation shielding jig 10E, and FIG. 3 shows the front view.

In the fifth Embodiment of radiation shielding jig 10E, the radiation shielding jig 10B (not shown) of the second Embodiment is further provided with a bulkhead 16 with ventilation holes that divide the inside of the tare 11 into round slices and a bulkhead 17 with ventilation holes that divide the inside of the tare 11 into layers.

The fifth Embodiment of the radiation shielding jig 10E makes it possible to prevent deformation of the tare 11 and movement of the shielding material particles 18 due to its own weight, and also prevents large movement of the shielding material particles 18 across the layers.

Therefore, each space portion divided by a bulkhead 16 and a bulkhead 17 can be filled with shielding material particles with the desired mixing ratio of sintered body particles and resin particles, thus the radiation shielding jig 10E with the desired radiation shielding performance can be provided.

According to the radiation shielding jig 10E, all of the above problems (1) through (5), which are required between the irradiated beam after emission and the patient affected area that is the target of the beam, and the above first problem through the above third problem regarding shielding performance when LiF-containing polyethylene resin is used as shielding material, can be solved reliably.

[Method for Using the Radiation Shielding Jig.]

The method for using the radiation shielding jig in BNCT will be explained using the case in which the radiation shielding jig 10E of the fifth Embodiment is used as the radiation shielding jig.

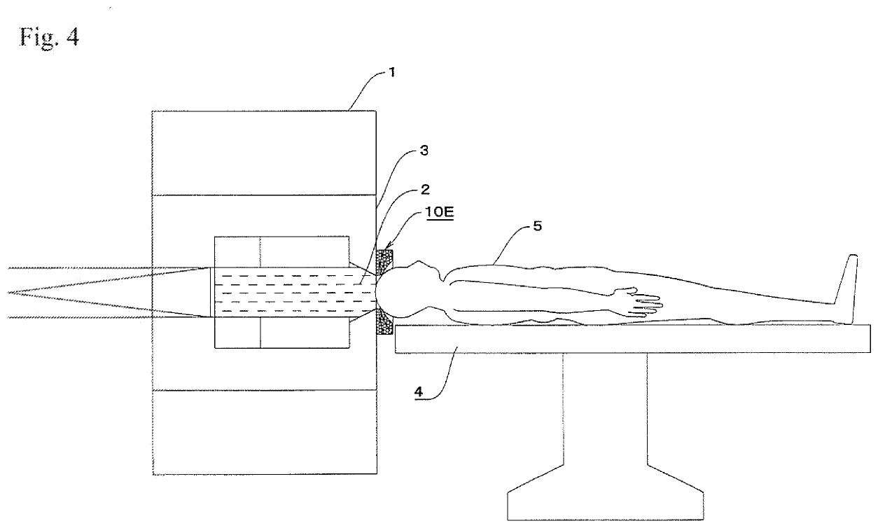

FIG. 4 is a partial cross-sectional side view of the system including the deceleration system of the BNCT device to illustrate the method for using the radiation shielding jig in BNCT.

A radiation shielding jig 10E is placed at the outer edge of the collimator 3 that constitutes the irradiation port of the therapeutic beam 2 of the BNCT device 1, in the form of a ring, i.e., a hollow three-dimensional shape, with an adjustment function for the amount of atmospheric gas inside it and a structure that allows its external shape to be fixed.

The radiation shielding jig 10E is filled with the shielding material particles 18, which are composed of sintered and resin particles of having the excellent radiation shielding performance and of a shape that has excellent flowability, to a predetermined packing density.

Based on the treatment plan, while opening the sealing valve 13 of the ventilation pipe 14 to which the gas suction pump 15 is connected, and close the other sealing valves 13.

While placing the patient's affected area of patient 5 lying on the treatment table 4 to the tare 11 with no gap in the treatment position, the gas suction pump 15 connected to the tare 11 shown in FIGS. 2 and 3 is operated.

While simultaneously adjusting the opening degree of the sealing valve 13 of the ventilation pipe 14, and when the planned setting condition is reached, the sealing valve 13 is fully closed and the gas suction pump 15 is stopped to fix the external shape of the tare 11.

This state is referred to as the “temporary setting state”.

Next, the patient's affected area is moved and the external shape of the tare 11 in the “temporary setting” is measured using a measurement method such as, for example, a laser scanner method, and the shape data is incorporated into a treatment plan consisting of a radiation behavior calculation code (Particle and Heavy Ion Transport code System: hereinafter referred to as PHITS)method, etc., and the treatment plan is made into an implementation version.

Treatment planning using this method is more accurate than treatment planning using three-dimensional shape data of the patient's affected area obtained from CT scans, MRI measurements, etc. in the current examination phase, because it uses actual measured shape data.

After such a preparatory stage of treatment, the patient's affected area is returned to the “temporary setting state” again, and this is the “setting state” based on the treatment plan, from which high-precision treatment by irradiation of the therapeutic beam 2 begins.

As described above, the radiation shielding jig 10E allows the external shape of the tare 11 to be “setting state” for different patients repeatedly by adjusting the amount of atmospheric gas in the tare 11.

Moreover, both the tare 11 and the shielding material particles 18 filled inside it are highly radiation resistant and can be used repeatedly.

When the radiation shielding jig 10E is used repeatedly, such as for another patient, a cloth cover tare made of non-woven fabric of the same material as the tare 11 is prepared separately from the tare 11 for sanitary reasons.

This cover tare is used over the tare 11, and only this cover tare should be replaced for each patient.

By making the radiation shielding jig 10E repeatable, the cost ratio of the radiation shielding jig 10E to the treatment cost can be significantly reduced. It can greatly contribute to the promotion of the product in the market.

### EXAMPLE

The following is description of examples of a radiation shielding jig.

The radiation shielding jig concerning the present invention is a jig used in direct contact with a patient, i.e., the human body, and it is difficult to collect data on shielding performance under actual conditions of use.

Therefore, we decided to evaluate its shielding performance in a simulated test instead.

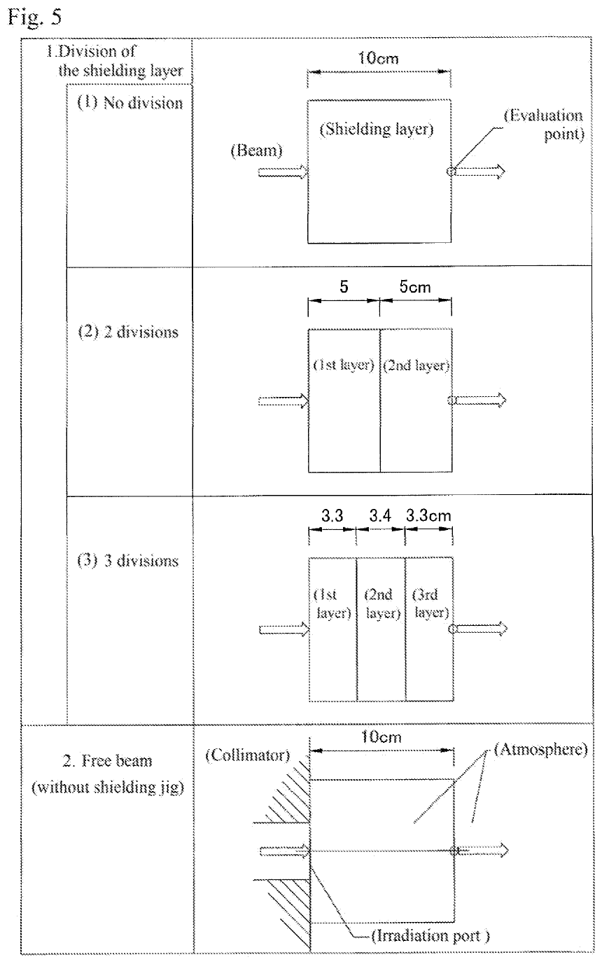

The simulated test on the shielding performance evaluation was based on the value of neutron flux when the irradiation beam flows through the irradiation port into the atmosphere without the “radiation shielding jig,” i.e., leaks through the atmosphere, as shown in FIG. 5, and the shielding performance of several different types of “radiation shielding jigs” in different forms was compared.

The shielding performance of the “radiation shielding jig” was determined by simulation calculations of Monte Carlo transport analysis using the PHITS method on a supercomputer for, the case where the shielding layer was not divided and the entire shielding material particle filling layer was a uniform filling layer, the case where the shielding layer was divided into two or three layers, the case where the mixture ratio of the sintered body particles and resin particles was changed, and the case where the concentration of the radioactive isotope elements ⁶Li, ¹⁰B and ¹⁵⁷Gd with shielding performance in the sintered particles was changed.

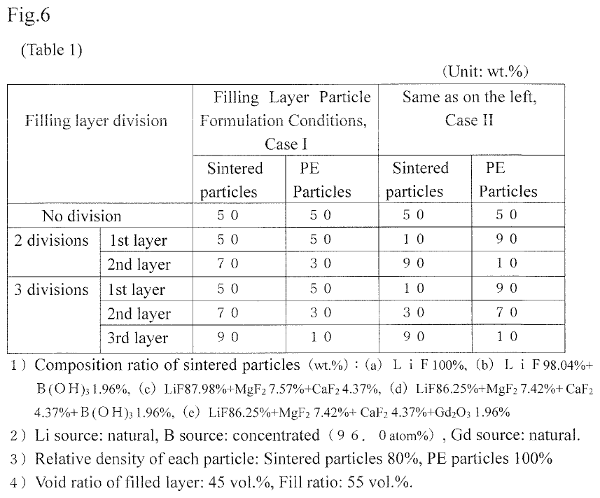

Specific simulation calculations were performed under the particle blending conditions for the shielding layer shown in FIG. 6 (Table 1), assuming a distance of 10 cm over which the irradiation beam flowed through the radiation shielding jig.

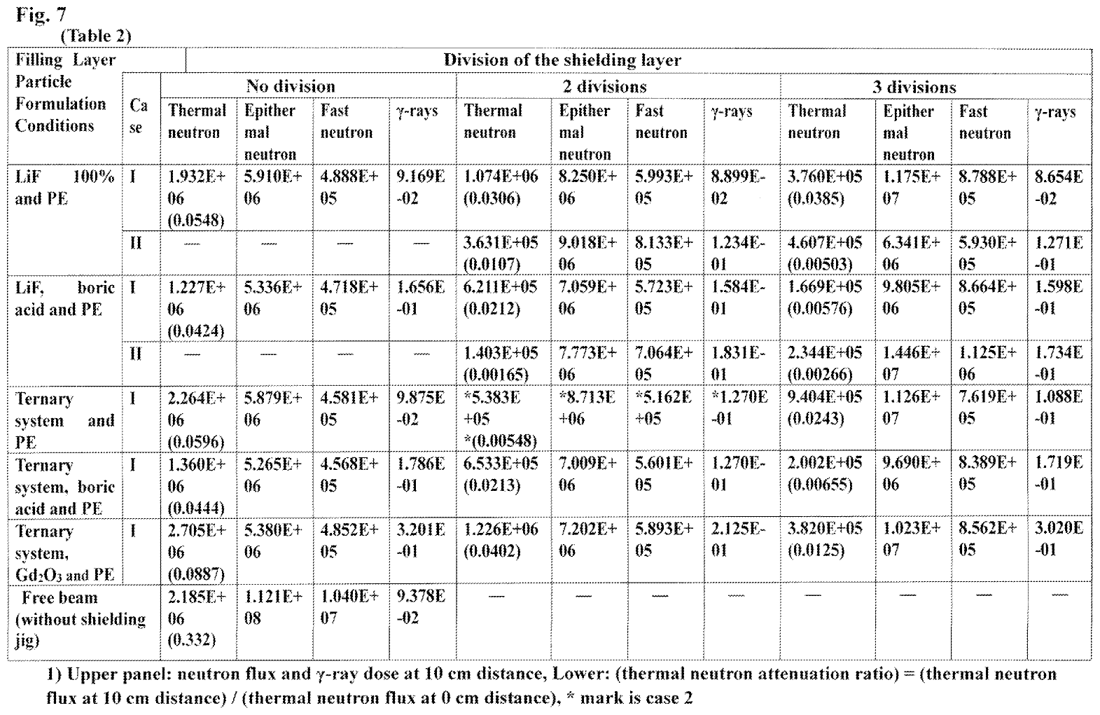

The shielding performance was compared by comparing the neutron flux (i.e., the amount of neutrons) at a distance of 10 cm from where the irradiation beam flowed for the base “without radiation shielding jig” case described above, and the “with radiation shielding jig” case of various conditions. The results of this analysis are shown in FIG. 7 (Table 2).

The most noteworthy result of this analysis is the “change in thermal neutron flux”. In particular, the ratio of the change in thermal neutron flux in the irradiated beam before and after the radiation shielding jig, i.e., the “attenuation ratio (=(thermal neutron flux at 10 cm distance)/(thermal neutron flux at 0 cm distance))” is large and small in the case of “with radiation shielding”.

The thermal neutron flux originally present in the irradiated beam is added to the thermal neutrons generated by the shielding and moderation of high-energy neutrons such as fast neutrons by the shielding material particles in the radiation shielding jig, and then subtracted by shielding by the shielding material particles in the radiation shielding jig: (thermal neutron flux at 10 cm distance).

The “attenuation ratio” is obtained by dividing (thermal neutron flux at 10 cm distance) by the thermal neutron flux in the irradiated beam (i.e., the thermal neutron flux at 0 cm). The “attenuation ratio” is an index representing the typical shielding performance of the shielding material particles according to the present invention.

The first point of interest is the comparison of the “attenuation ratio of thermal neutrons” between “free beam (without radiation shielding jig)” and “with radiation shielding jig”, which is the most important comparison.

Compared to the “free beam (without radiation shielding jig),” the attenuation ratio of thermal neutrons in the “with radiation shielding jig” was one or two orders of magnitude lower in all cases, regardless of whether the shielding layer was divided into two or three layers. It was confirmed that the shielding performance of the “with radiation shielding jig” was extremely excellent.

The second point of interest is the comparison of shielding performance by “with and without shielding layer division”, regardless of the particle composition conditions of the filling layer, in all cases the attenuation ratio of thermal neutrons is lower with division than without division, it was confirmed that “with shielding layer division” provides better thermal neutron shielding performance.

The third point of interest is the change in “extra-thermal neutron flux”, “fast neutron flux” and “γ-ray dose” between “free beam (without radiation shielding jig)” and “with radiation shielding jig”.

In the case of “with radiation shielding jig”, both “extra-thermal neutron flux” and “fast neutron flux” are one or two orders of magnitude lower than in the case of “free beam (without radiation shielding jig)”, it was confirmed that the radiation shielding jig according to the present invention has excellent neutron shielding performance.

On the other hand, in some cases, the change in the “γ-ray” dose was slightly increased, which was attributed to the generation of secondary radiation such as γ-rays due to the shielding reaction in the shielding layer.

However, the increase in their “γ-ray” doses has not reached the point where it becomes a problem, it was confirmed that the radiation shielding jig according to the present invention has overall excellent neutron shielding performance.

In addition to having excellent radiation shielding performance, the radiation shielding jig according to the present invention can improve the effectiveness of treatment by improving the positioning accuracy of the patient's affected area.

Reduction of the pressure the patient receives from the treatment device, prevention of exposure of patients and medical personnel, and reduction of the activation of peripheral equipment, and so on, the radiation shielding jig according to the present invention is an extremely superior radiation shielding jig that exhibits a number of excellent effects.

### DESCRIPTION OF REFERENCE SIGNS

1: BNCT device
2: Therapeutic beam
3: Collimator
4: Treatment table
5: Patient
10A, 10E: Radiation shielding jig
11: Tare
11r: Radiation pathway portion
13: Sealing valve
14: Ventilation pipe
15: Gas suction pump
16: Bulkhead (round slices)
17: Bulkhead (layered)
18: Shielding material particles

1. A radiation shielding jig comprising a tare filled with shielding material particles;
the tare is made of a resin fabric and has a hollow three-dimensional shape with a radiation pathway portion,
the shielding material particles comprising a mixture of sintered particles having a predetermined particle diameter with radiation shielding performance and resin particles having a predetermined particle diameter.

2. Radiation shielding jig according to claim 1, wherein:
at least one ventilation tube with a sealing valve that can be connected to a gas suction pump is connected to the tare.

3. Radiation shielding jig according to claim 1, wherein:
having at least one bulkhead with ventilation holes that divides the interior space of the tare into round slices.

4. Radiation shielding jig according to claim 1, wherein:
having at least one bulkhead with ventilation holes that divides the interior space of the tare into layers.

5. Radiation shielding jig according to claim 3, wherein:
having at least one bulkhead with ventilation holes that divides the interior space of the tare into layers.

6. Radiation shielding jig according to claim 1, wherein:
the tare and the resin particles are made of resin selected from polyethylene, polystyrene, and polypropylene.

7. Radiation shielding jig according to claim 5, wherein:
the bulkhead is made of a resinous fabric selected from polyethylene, polystyrene, and polypropylene.

8. Radiation shielding jig according to claim 1, wherein:
the particle size of the sintered particles and the resin particles are set in the range of 0.5 mm to 7 mm.

9. Radiation shielding jig according to claim 1, wherein:
the sintered particles are collected by fracturing and abrasion fracturing and sieving sintered body with a relative density of 70-90%.

10. Radiation shielding jig according to claim 9, wherein:
the sintered particles are collected from LiF sintered body.

11. Radiation shielding jig according to claim 9, wherein:
the sintered particles are collected from a mixed system sintered body consisting of LiF with a boron compound 0.1-5 wt. % as boron isotope ¹⁰B, wherein a boron compound is selected from B₂O₃, B(OH)₃, BF₃, LiB₃O₅ or Li₂B₄O₇.

12. Radiation shielding jig according to claim 9, wherein:
the sintered particles are collected from multicomponent system fluoride sintered body with LiF as a main phase, wherein:
multicomponent system fluoride sintered body containing 99 wt. % to 5 wt. % of LiF and 1 wt. % to 95 wt. % of one or more fluorides selected from MgF₂, CaF₂, AlF₃, KF, NaF and/or YF₃.

13. Radiation shielding jig according to claim 9, wherein:
the sintered particles are collected from a mixed system sintered body consisting of multicomponent system fluoride with LiF as a main phase and boron compounds selected from B₂O₃, B(OH)₃, BF₃, LiB₃O₅ or Li₂B₄O₇, with 0.1 to 5 wt. % as boron isotope ¹⁰B added.

14. Radiation shielding jig according to claim 9, wherein:
the sintered particles are collected from a mixed system sintered body consisting of multicomponent system fluoride with LiF as a main phase and a gadolinium compound selected from Gd₂O₃, Gd(OH)₃ or GdF₃, with 0.1 to 2 wt. % as gadolinium isotope ¹⁵⁷Gd added.

15. Radiation shielding jig according to claim 9, wherein:
the sintered particles are collected from a mixed system sintered body consisting of multicomponent system fluoride with LiF as a main phase, and boron compounds selected from B₂O₃, B(OH)₃, BF₃, LiB₃O₅ or Li₂B₄O₇, with 0.1 to 5 wt. % as boron isotope ¹⁰B added, and a gadolinium compound selected from Gd₂O₃, Gd(OH)₃ or GdF₃, with 0.1 to 2 wt. % as gadolinium isotope ¹⁵⁷Gd added.

16. Radiation shielding jig according to claim 9, wherein:
the sintered particles of which are formed by fracturing, abrasion fracturing, and sieving the sintered body, are not collected particles,
using the not collected particles, the sintered particles are formed by re-pulverizing, mixing with the raw powder, and re-sintering.

17. Radiation shielding jig according to claim 1, wherein:
the mixing ratio of the sintered particles and the resin particles in the shielding material particles is set between 10 wt. % and 90 wt. %,
the angle of repose of the shielding material particles is from 8 to 45 degrees of fluidity.

18. Radiation shielding jig according to claim 4, wherein:
the upstream portion of the beam flow of the tare, which is divided into layers, is filled with the sintered particles in a ratio of not less than 10 wt. % and not more than 50 wt. % and the resin particles in a ratio of not less than 50 wt. % and not more than 90 wt. %,
on the other hand, the downstream portion of the beam flow of the tare is filled with the sintered particles in a ratio of not less than 50 wt. % and not more than 90 wt. % and the resin particles in a ratio of not less than 10 wt. % and not more than 50 wt. %.

19. The method for manufacturing a radiation shielding jig according to claim 1, comprising the steps of:
fracturing the sintered body using a fracturing machine and abrasion fracturing using a abrasion fracturing machine, and sieving, collecting the sintered particles of a predetermined particle size;
mixing the collected sintered particles with resin particles of a predetermined particle size in a predetermined ratio; and
filling the mixed sintered particles and the resin particles into the tare.

20. The method for using a radiation shielding jig according to claim 1, comprising the steps of:
placing the patient's affected area to the tare with no gap in the treatment position; and
initiating radiation therapy then.

21. The method for using a radiation shielding jig according to claim 2, comprising the steps of:
opening the sealing valve of the ventilation pipe to which the gas suction pump is connected, and closing the other sealing valves, and placing the patient's affected area to the tare with no gap in the treatment position, while operating the gas suction pump;
in this placing state, closing the sealing valve of the ventilation pipe to which the gas suction pump is connected to fix the external shape of the tare; and
initiating radiation therapy then.

22. The method for using a radiation shielding jig according to claim 2, comprising the steps of:
opening the sealing valve of the ventilation pipe to which the gas suction pump is connected, and closing the other sealing valves, and placing the patient's affected area to the tare with no gap in the treatment position, while operating the gas suction pump;
in this placing state, closing the sealing valve of the ventilation pipe to which the gas suction pump is connected to fix the external shape of the tare;
measuring the external shape of the fixed tare then;
performing simulation calculations on the behavior of the irradiation beam during treatment based on the measured external shape data of the tare then;
creating a treatment plan based on the results of this simulation calculation; and
performing radiation therapy according to the treatment plan.
